(19) United States
(12) Patent Application Publication
(10) Pub. No.: US 2017/0135948 A1
(43) Pub. Date: May 18, 2017

(54) COMPOSITION COMPRISING EXTRACT OF AUTUMN SOYBEAN LEAVES

(71) Applicant: AMOREPACIFIC CORPORATION, Seoul (KR)

(72) Inventors: Young Gyu KANG, Yongin-si, Gyeonggi-do (KR); Ok Chan LEE, Yongin-si, Gyeonggi-do (KR); Yong Deog HONG, Yongin-si, Gyeonggi-do (KR); Myeong Hun YEOM, Yongin-si, Gyeonggi-do (KR); Jun Seong PARK, Yongin-si, Gyeonggi-do (KR)

(21) Appl. No.: 15/320,012

(22) PCT Filed: Jun. 18, 2015
(86) PCT No.: PCT/KR2015/006175

§ 371 Date: Dec. 19, 2016

(30) Foreign Application Priority Data
Jun. 19, 2014 (KR) 10-2014-0074820
Jun. 16, 2015 (KR) 10-2015-0085091

(51) Int. Cl. A61K 8/97; A61Q 19/00; A61K 36/48; A61K 9/00; A23L 33/105; A61K 9/20; A61K 9/14; A61K 9/06; A23L 2/52; A61Q 19/08; A61K 9/48

(52) U.S. Cl. CPC A61K 8/97; A61Q 19/08; A61Q 19/007; A61K 36/48; A61K 9/0014; A61K 9/48; A61K 9/20; A61K 9/14; A61K 9/0019; A61K 9/06; A23L 2/52; A23L 33/105; A23V 2002/00

(57) ABSTRACT

The present specification relates to a composition for a skin preparation for external use, containing an extract of bean leaves (danpung bean leaves) of a specific stage among the growth stages of beans as an active ingredient. The composition according to the present invention has an excellent antioxidant property, an excellent antiaging property and no skin irritation since natural-derived ingredients are contained in the composition, and thus is useful. The composition of the present specification can be widely used, by such characteristics, as an antioxidant or antiaging composition of an individual in the pharmaceutical or cosmetic field.

### TECHNICAL FIELD

The present disclosure relates to a composition containing an extract of the leaf of soybean during a particular growth stage.

### BACKGROUND ART

Human skin experiences changes with aging due to various intrinsic and extrinsic factors. Intrinsically, the secretion of various hormones regulating metabolism decreases and the function and activity of immune cells decline. As a result, the biosynthesis of immunoproteins and constituent proteins decreases. Extrinsically, as the amount of ultraviolet light reaching the surface of the earth increases due to the destruction of the ozone layer and environmental pollution is aggravated, free radicals, reactive oxygen species, etc. are increased. As a result, various changes occur, including decreased skin thickness, increased wrinkles, decreased skin elasticity, darkening of skin color, frequent occurrence of skin troubles, increased freckles, live spots and blotches, etc.

As aging proceeds, skin experiences change or decrease in the contents and arrangement of the substances constituting the skin, such as collagen, elastin, hyaluronic acid and glycoproteins, and oxidative stresses are caused by free radicals and reactive oxygen species. Also, it is known that aging or UV light increases the biosynthesis of cyclooxygenase-2 (COX-2), which is an enzyme that produces proinflammatory cytokines known to cause inflammations in most cells constituting skin, the biosynthesis of matrix metalloproteinase (MMP), which is an enzyme that degrades skin tissues, and the production of nitric oxide (NO) by inducible nitric oxide synthase (iNOS). That is to say, as the biosynthesis of substrate materials is decreased due to decreased cellular activity and microinflammations with natural intrinsic aging and their degradation is accelerated due to extrinsic factors such as increased stresses caused by various harmful environments and increase in reactive oxygen species, many symptoms of skin aging occur including the destruction and thinning of the skin. Accordingly, many studies are ongoing on active ingredients capable of preventing and improving the aging phenomena.

The reactive oxygen species which are produced due to various physical, chemical and environmental factors, including enzymatic reactions, reductive metabolism, chemicals, pollutants, photochemical reactions, etc., are known to cause cellular aging and various diseases including cancer by non-selectively and irreversibly destroying the cellular components such as lipids, proteins, sugars, DNAs, etc. In addition, various peroxides including lipid peroxides produced by lipid peroxidation by these reactive oxygen species cause a variety of diseases by inducing functional disorders through oxidative damage of cells. Therefore, antioxidants such as the compounds that can scavenge the free radicals (free radical scavengers) or the substances that can inhibit the production of peroxides are viewed as candidates for prevention or treatment of aging and various diseases caused by the oxides.

The inventors of the present disclosure have found out that a soybean leaf extract can improve not only the antioxidative and antiaging functions but also the skin-whitening and skin-moisturizing functions of skin when applied to the skin and have completed the present disclosure.

### DISCLOSURE

### Technical Problem

The present disclosure is directed to providing a naturally derived composition which improves skin health.

### Technical Solution

In an aspect, the present disclosure provides a composition containing an autumn soybean leaf extract as an active ingredient.

### Advantageous Effects

A composition according to the present disclosure exhibits superior antioxidative activity and antiaging activity and is without skin irritation because it contains naturally derived ingredients. Due to these characteristics, the composition of the present disclosure can be widely used in the field of pharmacy or cosmetics as an antioxidative or antiaging composition for individuals.

### BRIEF DESCRIPTION OF DRAWINGS

FIG. 1 shows an HPLC analysis result for an autumn soybean leaf extract according to an aspect of the present disclosure and a soybean extract.

### BEST MODE

Korean Patent Application No. 10-2014-0074820 filed on Jun. 19, 2014 and Korean Patent Application No. 10-2015-0085091 filed on Jun. 16, 2015 are incorporated in the present disclosure in their entirety for all purposes. Also, this application claims all the benefits of Korean Patent Application Nos. 10-2014-0074820 and 10-2015-0085091.

In an aspect, the present disclosure may relate to a composition containing an autumn soybean leaf extract as an active ingredient. Specifically, in an aspect of the present disclosure, the composition may be a composition which contains an extract of the leaf of autumn soybean which has turned yellow as an active ingredient. More specifically, in an aspect of the present disclosure, the autumn soybean leaf may be the leaf of soybean in growth stages R7 to R8.

In an aspect of the present disclosure, the composition may be a composition for external application to skin.

In an aspect of the present disclosure, the composition may be an antioxidative or antiaging composition.

In an aspect of the present disclosure, the composition may be a pharmaceutical, cosmetic or food composition.

In another aspect, the present disclosure may relate to a method for antioxidation, which includes administering an autumn soybean leaf extract or a composition containing the same as an active ingredient to a subject in need of antioxidation. Specifically, the administration may be achieved by an administration method described in the present disclosure.

In another aspect, the present disclosure may relate to a use of an autumn soybean leaf extract for antioxidation.

In another aspect, the present disclosure may relate to an autumn soybean leaf extract for use in antioxidation.

In another aspect, the present disclosure may relate to a method for antiaging, which includes administering an autumn soybean leaf extract or a composition containing the same as an active ingredient to a subject in need of antiaging. Specifically, the administration may be achieved by an administration method described in the present disclosure.

In another aspect, the present disclosure may relate to a use of an autumn soybean leaf extract for antiaging.

In another aspect, the present disclosure may relate to an autumn soybean leaf extract for use in antiaging.

In an aspect of the present disclosure, the autumn soybean leaf extract or the composition containing the same as an active ingredient may have one or more of the following characteristics:

(1) IC₅₀≦70 ppm in antioxidative activity test based on DPPH reduction; and

(2) collagenase expression level≦55% in collagenase production inhibition test.

In another aspect, the present disclosure may relate to an autumn soybean leaf extract or a composition containing the same as an active ingredient, which has one or more of the following characteristics:

(1) IC₅₀≦70 ppm in antioxidative activity test based on DPPH reduction; and

(2) collagenase expression level≦60% in collagenase production inhibition test.

In an aspect of the present disclosure,

(1) the IC₅₀ in antioxidative activity test based on DPPH reduction may be 70 ppm or lower, 65 ppm or lower, 60 ppm or lower, 55 ppm or lower, 50 ppm or lower, 48 ppm or lower, 46 ppm or lower, 44 ppm or lower, 42 ppm or lower, 41 ppm or lower, 40 ppm or lower, 38 ppm or lower, 36 ppm or lower, 34 ppm or lower, 30 ppm or lower, 25 ppm or lower, 20 ppm or lower, 15 ppm or lower, 10 ppm or lower, 5 ppm or lower or 1 ppm or lower; and

(2) the collagenase expression level in collagenase production inhibition test may be 60% or lower, 58% or lower, 56% or lower, 55% or lower, 54% or lower, 53% or lower, 52% or lower, 51% or lower, 50% or lower, 48% or lower, 46% or lower, 44% or lower, 42% or lower, 40% or lower, 35% or lower, 30% or lower, 20% or lower, 10% or lower, 5% or lower or 1% or lower.

In the present disclosure, the “autumn soybean leaf” may refer to a soybean leaf which has turned yellow. Specifically, it means the leaf of soybean in 1) a growth stage where the bean hull and bean begin to turn yellow (hereinafter, stage R7) or 2) a growth stage where the leaf has fallen and the bean hull and bean have turned yellow completely (hereinafter, stage R8). Specifically, the “autumn soybean leaf” may be a soybean leaf which is turning from green to yellow or which has turned yellow completely. It also includes a soybean leaf which has turned yellow partly.

In the present disclosure, ‘antioxidation’ refers to an effect of delaying or preventing oxidation processes known in the art without limitation.

In the present disclosure, ‘antiaging’ refers to an effect of delaying or preventing aging processes known in the art. Specifically, it may mean an effect of enhancing skin elasticity and improving wrinkles by effectively inhibiting the expression of collagenase in skin and thereby reducing collagen degradation in the skin, although not being limited thereto.

In an aspect of the present disclosure, the composition may exhibit an activity of inhibiting DPPH oxidation and an effect of inhibiting the expression of collagenase or inhibiting the activity of collagenase. Specifically, in an aspect of the present disclosure, the composition may be a composition for inhibiting DPPH oxidation or a composition for inhibiting the expression or activity of collagenase.

In an aspect of the present disclosure, the composition may contain the autumn soybean leaf extract in an amount of 0.1 μg/mL or more, 1 μg/mL or more, 5 μg/mL or more, 10 μg/mL or more, 20 μg/mL or more, 30 μg/mL or more, 50 μg/mL or more, 70 μg/mL or more, 80 μg/mL or more, 90 μg/mL or more, 100 μg/mL or more, 110 μg/mL or more, 120 μg/mL or more, 130 μg/mL or more, 150 μg/mL or more, 170 μg/mL or more, 200 μg/mL or more, 500 μg/mL or more or 1000 μg/mL or more, or 2000 μg/mL or less, 1000 μg/mL or less, 500 μg/mL or less, 300 μg/mL or less, 200 μg/mL or less, 170 μg/mL or less, 150 μg/mL or less, 130 μg/mL or less, 120 μg/mL or less, 110 μg/mL or less, 100 μg/mL or less, 90 μg/mL or less, 80 μg/mL or less, 70 μg/mL or less, 50 μg/mL or less, 30 μg/mL or less, 20 μg/mL or less, 10 μg/mL or less, 5 μg/mL or less, 1 μg/mL or less or 0.1 μg/mL or less, based on the total volume of the composition.

In an aspect of the present disclosure, the composition may contain the autumn soybean leaf extract in an amount of 0.001 wt % or more, 0.01 wt % or more, 0.1 wt % or more, 0.5 wt % or more, 1 wt % or more, 2 wt % or more, 3 wt % or more, 4 wt % or more, 5 wt % or more, 6 wt % or more, 7 wt % or more, 8 wt % or more, 9 wt % or more, 10 wt % or more, 15 wt % or more or 20 wt % or more, or 20 wt % or less, 15 wt % or less, 10 wt % or less, 9 wt % or less, 8 wt % or less, 7 wt % or less, 6 wt % or less, 5 wt % or less, 4 wt % or less, 3 wt % or less, 2 wt % or less, 1 wt % or less or 0.1 wt % or less, based on the total weight of the composition.

The autumn soybean leaf extract of the present disclosure may be obtained, for example, by extracting and depositing washed, dried and grounded autumn soybean leaf powder in water or an organic solvent, separating a filtrate from a residue through filtration through filter cloth and centrifugation and then concentrating the separated filtrate under reduced pressure. In an aspect of the present disclosure, the organic solvent that can be used for the extraction may be selected from ethanol, methanol, butanol, ether, ethyl acetate or chloroform. Also, a mixture solvent of the organic solvent with water may be used. Specifically, water or 30-70% ethanol may be used in consideration of the safety of the raw material. The extract obtained using the solvent may be precipitated at room temperature, heated and filtered according to a common method known in the art to obtain a liquid or the solvent may be further evaporated, spray-dried or freeze-dried. However, without being limited thereto, any method commonly used in the art may be used without limitation.

In an aspect of the present disclosure, the autumn soybean leaf extract may be extracted from the leaf of soybean in growth stages R7 to R8. In the present disclosure, the growth stages of soybean are as follows.

Stage VE: 1-2 weeks after seeding. Cotyledons emerge from the soil.

Stage VC: The cotyledons spread out and outer leaves are produced as one node is formed.

Stage V1: Three leaves are produced as one node is formed from first outer leaves.

Stage V2: Three leaves are produced as one additional node is formed in Stage V1.

Stage V3: Three leaves are produced as one additional node is formed in Stage V2.

Stage V4: Three leaves are produced as one additional node is formed in Stage V3.

Stage R2: Full bloom.

Stage R4: Full bean hull.

Stage R5: Seeds are formed in the bean hull.

Stage R6: Green seeds are completed in the bean hull.

Stage R7: The bean hull and the seeds turn yellow.

Stage R8: The bean hull and the seeds have turned yellow completely as the leaves fall.

In an aspect of the present disclosure, the ‘soybean’ is not limited in its kind. For example, it may be one or more selected from a group consisting of seoritae (Glycine max Merr.), seomoktae (Rhynchosia nulubilis), black soybean (Glycine max (L.) Merr.), blue bean (Glycine max Merr.), yellow bean (Glycine max Merr.), field bean (Vicia faba), kidney bean (Phaseolus vulgaris), pinto bean (Phaseolus vulgaris L.), small red bean (Vigna angularis), small black bean (Phaseolus angularis W.F. Wight), sprouting bean (Glycine max (L.) Merr.) and soybean (Glycine max), although not being limited thereto.

In an aspect of the present disclosure, the ‘soybean leaf’ refers to the leaf of soybean.

In an aspect of the present disclosure, the “extract” refers to a substance extracted from a natural product, regardless of the extraction method, extraction solvent, extracted components or the type of the extract. It is used in a broad concept, including any substance that can be obtained by processing or treatment of a substance extracted from a natural product. Specifically, the processing or treatment may be additionally fermenting or enzymatically treating the extract. Accordingly, in the present disclosure, the extract is used in a broad concept, including a fraction product, a fermentation product, a concentrate and a dried product. Specifically, in the present disclosure, the extract may be a fermentation product.

In an aspect of the present disclosure, the “autumn soybean leaf extract” includes any substance extracted from the autumn soybean leaf, regardless of the extraction method, extraction solvent, extracted components or the type of the extract. It includes a substance obtained by an extraction method including a process treating with heat, an acid, a base, an enzyme, etc. and is used in a broad concept, including any substance that can be obtained by processing or treatment of a substance extracted from the autumn soybean leaf. Specifically, the processing or treatment may be additionally fermenting or enzymatically treating the autumn soybean leaf extract. In an aspect of the present disclosure, the autumn soybean leaf extract may be a fermentation product.

In an aspect of the present disclosure, the “autumn soybean leaf” may be an extract, a live autumn soybean leaf, a pulverization product of live autumn soybean leaf, a dried product of live autumn soybean leaf, a dried pulverization product of live autumn soybean leaf or a fermentation product of autumn soybean leaf, although not being limited thereto. And, the autumn soybean leaf used in the present disclosure is not limited in the way how it is obtained. It may be either cultivated or purchased commercially. Also, all or part of herbal areal part or root part may be used. More specifically, one or more selected from a group consisting of the leaf, stem, root and flower of the herb may be used. More specifically, the flower, leaf and stem may be used. The autumn soybean leaf of the present disclosure is not necessarily dried and may be in any form as long as the active ingredients of autumn soybean leaf can be adequately extracted.

In an aspect of the present disclosure, the autumn soybean leaf extract may be an extract of one or more selected from a group consisting of water, an organic solvent and a mixture thereof.

In an aspect of the present disclosure, the water includes distilled water or purified water, and the organic solvent includes one or more selected from a group consisting of a C₁-C₆ lower alcohol, acetone, ether, ethyl acetate, diethyl ether, ethyl methyl ketone and chloroform, although not being limited thereto. Specifically, the alcohol may be methanol or ethanol.

In an aspect of the present disclosure, the autumn soybean leaf extract may be an extract obtained by sequentially extracting with ethanol and ethyl acetate.

In an aspect of the present disclosure, the autumn soybean leaf extract may include an ethyl acetate extract of autumn soybean leaf.

In an aspect of the present disclosure, the autumn soybean leaf extract or the composition containing the same as an active ingredient may be obtained by a method including a step of extracting autumn soybean leaf with water, an organic solvent or a mixture thereof. Specifically, in an aspect of the present disclosure, the preparation method may include: a step of extracting autumn soybean leaf first with water, an organic solvent or a mixture thereof; a step of extracting second by adding an organic solvent to the resulting extract; and a step of concentrating the resulting extract under reduced pressure and then drying the same.

In an aspect of the present disclosure, the autumn soybean leaf extract or the composition containing the same as an active ingredient may be prepared by the preparation method according to an aspect of the present disclosure.

In another aspect, the present disclosure may relate to a method for preparing an autumn soybean leaf extract or a composition containing the same as an active ingredient, which includes a step of extracting autumn soybean leaf with water, an organic solvent or a mixture thereof.

In an aspect of the present disclosure, the method may further include, before the extraction step, a step of washing soybean leaf with purified water and then drying and grinding the same.

In an aspect of the present disclosure, the method may further include, after the extraction step, a step of extracting again (second extraction) by adding an organic solvent. Specifically, in the second extraction step, an upper layer (organic solvent layer) may be taken after the extracted solution is separated into two layers. In this case, the preceding extraction step is called a first extraction step.

In an aspect of the present disclosure, the second extraction step may be repeated one or more times, two or more times or three more times for a given solution. Specifically, after taking the separated upper layer (organic solvent layer), the same procedure may be repeated for the lower layer (aqueous layer) by adding an organic solvent again. Specifically, the organic solvent may be ethyl acetate.

In an aspect of the present disclosure, the method may further include, after the second extraction step, a step of concentrating the extract under reduced pressure and drying the same.

In an aspect of the present disclosure, the autumn soybean leaf extract may be a crude extract of a solvent selected from a group consisting of water, an organic solvent and a combination thereof. The organic solvent may be a C₁-C₆ alcohol. Specifically, the C₁-C₆ alcohol may be methanol or ethanol. In an aspect of the present disclosure, when the autumn soybean leaf is extracted with a solvent, the solvent may be added in an amount of about 5-15 times that of the autumn soybean leaf. Specifically, the solvent may be added in an amount of about 10 times that of the autumn soybean leaf, although not being limited thereto.

In an aspect of the present disclosure, the extraction may be performed by hot water extraction, ethanol extraction, heating extraction, cold extraction, reflux extraction, reflux condensation extraction, ultrasonic extraction, etc. Any extraction method known to those skilled in the art may be used without limitation. Specifically, the extraction may be performed by hot water extraction or ethanol extraction.

In an aspect of the present disclosure, the extraction may be performed at room temperature. However, for more effective extraction, it may be performed at elevated temperatures. The extraction may be performed specifically at about 40-100° C., more specifically at about 80° C., although not being limited thereto. The extraction may be performed for about 2-14 hours, specifically for about 8-14 hours, more specifically for about 11-13 hours, most specifically for 12 hours. However, the extraction time is not limited thereto and may vary depending on such conditions as extraction solvent, extraction temperature, etc. The extraction may be performed more than once in order to obtain a larger amount of the active ingredients. The extraction may be performed continuously specifically 1-5 times, more specifically 3 times in succession, and the resulting extracts may be combined for further use.

In an aspect of the present disclosure, the autumn soybean leaf extract may include the crude extract of autumn soybean leaf as described above and may also include a soluble fraction of an organic solvent with low polarity, which is obtained by further extracting the crude extract with the organic solvent. In an aspect of the present disclosure, the organic solvent may be hexane, methylene chloride, ethyl acetate, n-butanol, etc., although not being limited thereto. The extract or the soluble fraction of the extract may be used as it is, may be used in form of a extract after being filtered and then concentrated, or may be used in form of a dried product after being concentrated and dried.

In an aspect of the present disclosure, the drying may be evaporation drying, spray drying or freeze drying. Specifically, freeze drying may be performed at −50 to −70° C. for 3-4 days.

Formulations of the cosmetic composition according to an aspect the present disclosure are not particularly limited but may be selected properly depending on purposes. For example, the composition may be prepared into one or more formulation selected from a group consisting of a skin lotion, a skin softener, a skin toner, an astringent lotion, a milk lotion, a moisturizing lotion, a nourishing lotion, a massage cream, a nourishing cream, a moisturizing cream, a hand cream, a foundation, an essence, a nourishing essence, a pack, a soap, a cleansing foam, a cleansing lotion, a cleansing cream, a body lotion and a body cleanser, although not being limited thereto.

When the cosmetic composition according to the present disclosure is formulated as a paste, a cream or a gel, an animal fiber, a plant fiber, wax, paraffin, starch, tragacanth, a cellulose derivative, polyethylene glycol, silicone, bentonite, silica, talc, zinc oxide, etc. may be used as a carrier component.

When the cosmetic composition according to the present disclosure is formulated as a powder or a spray, lactose, talc, silica, aluminum hydroxide, calcium silicate or polyamide powder may be used as a carrier component. In particular, when it is formulated as a spray, it may further contain a propellant such as chlorofluorohydrocarbon, propane/butane or dimethyl ether.

When the cosmetic composition according to the present disclosure is formulated as a solution or an emulsion, a solvent, a solubilizer or an emulsifier may be used as a carrier component. For example, water, ethanol, isopropanol, ethyl carbonate, ethyl acetate, benzyl alcohol, benzyl benzoate, propylene glycol, 1,3-butyl glycol oil, glycerol fatty ester, or fatty acid ester of polyethylene glycol or sorbitan may be used.

When the cosmetic composition according to the present disclosure is formulated as a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, etc. may be used as a carrier component.

When the cosmetic composition according to the present disclosure is formulated as a surfactant-containing cleanser, fatty alcohol sulfate, fatty alcohol ether sulfate, sulfosuccinic acid monoester, isethionate, an imidazolinium derivative, methyl taurate, sarcosinate, a fatty acid amide ether sulfate, an alkyl amidobetaine, a fatty alcohol, a fatty acid glyceride, a fatty acid diethanolamide, a vegetable oil, a lanolin derivative, an ethoxylated glycerol fatty acid ester, etc. may be used as a carrier component.

The cosmetic composition according to the present disclosure may further contain a functional additive and ingredients commonly used in a cosmetic composition, in addition to the autumn soybean leaf extract. The functional additive may include those selected from a group consisting of a water-soluble vitamin, an oil-soluble vitamin, a polypeptide, a polysaccharide, a sphingolipid and a seaweed extract.

If necessary, the cosmetic composition of the present disclosure may further contain, in addition to the functional additive, the ingredients commonly used in a cosmetic composition. Examples of the further contained ingredients may include an oil, a fat, a humectant, an emollient, a surfactant, an organic or inorganic pigment, an organic powder, a UV absorbent, a preservative, a sterilizer, an antioxidant, a plant extract, a pH control agent, an alcohol, a colorant, a flavor, a blood circulation promoter, a cooling agent, an antiperspirant, purified water, etc.

The present disclosure also relates to a composition for external application to skin, which contains an autumn soybean leaf extract as an active ingredient. The composition for external application to skin refers to any type of composition that can be applied from outside the skin and various types of cosmetics may be included therein.

The pharmaceutical composition according to the present disclosure may be in the form of various oral or parenteral formulations. When the composition is formulated, a commonly used diluent or excipient such as a filler, an extender, a binder, a wetting agent, a disintegrant, a surfactant, etc. is used. Solid formulations for oral administration include a tablet, a pill, a powder, a granule, a soft or hard capsule, etc., and these solid formulations may be prepared by mixing at least one excipient such as starch, calcium carbonate, sucrose, lactose, gelatin, etc. In addition to the simple excipient, a lubricant such as magnesium stearate, talc, etc. may also be used. Examples of liquid formulations for oral administration include a suspension, a liquid for internal use, an emulsion, a syrup, etc. In addition to a commonly used simple diluent such as water and liquid paraffin, various excipients such as a humectant, a sweetener, an aromatic, a preservative, etc. may also be contained. Formulations for parenteral administration include a sterilized aqueous solution, a non-aqueous solution, a suspension, an emulsion, a lyophilized product and a suppository. The non-aqueous solution or suspension may contain propylene glycol, polyethylene glycol, a vegetable oil such as olive oil, an injectable ester such as ethyl oleate, etc. As a base of the suppository, witepsol, macrogol, tween 61, cocoa butter, laurin butter, glycerogelatin, etc. may be used.

The composition of the present disclosure may contain a pharmaceutically acceptable salt of the active ingredient, and may be used alone or in combination with another pharmaceutically active compound(s). The salt is not particularly limited as long as it is pharmaceutically acceptable. For example, a salt of hydrochloric acid, sulfuric acid, nitric acid, phosphoric acid, hydrofluoric acid, hydrobromic acid, formic acid, acetic acid, tartaric acid, lactic acid, citric acid, fumaric acid, maleic acid, succinic acid, methanesulfonic acid, benzenesulfonic acid, toluenesulfonic acid, naphthalenesulfonic acid, etc. may be used.

The composition of the present disclosure may be administered parenterally or orally depending on the desired purposes. A daily dose of 0.1-500 mg, specifically 1-100 mg, per kg body weight may be administered once or several times a day. The administration dose for a particular patient may be changed depending on the body weight, age, sex and health conditions of the patient, diet, administration time, administration method, rate of excretion, severity of a disease, and so forth.

The pharmaceutical composition according to the present disclosure may be formulated into any pharmaceutically appropriate form including oral formulations such as a powder, a granule, a tablet, a soft or hard capsule, a suspension, an emulsion, a syrup, an aerosol, etc., formulations for external application to skin such as an ointment, a cream, etc., a suppository, an injection, a sterile solution for injection, etc. according to commonly employed methods. Specifically, it may be formulated into an injection or a formulation for external application to skin.

The composition according to the present disclosure may be administered to mammals including rat, mouse, cattle, human, etc. via various routes including parenteral and oral routes. All types of administration may be expected. For example, it may be administered orally, transdermally, rectally, intravenously, intramuscularly, subcutaneously, intrauterinally or intracerebroventricularly.

The composition according to the present disclosure may be administered via various routes that can be easily selected by those skilled in the art. In particular, the pharmaceutical composition according to the present disclosure may be applied on skin as a formulation for external application to skin.

Hereinafter, the present disclosure will be described in detail through examples and test examples. However, the following examples and test examples are for illustrative purposes only and the scope of the present disclosure is not limited by the examples.

### EXAMPLE 1

### Preparation of Soybean Leaf Extract

Soybean leaves harvested at different growth stages (stages VC, V2, V4, R2, R4, R6 and R7, 7 stages) were washed with purified water, dried and then ground into powder, repectively. 100 g of the soybean leaf powder was extracted with 1 L of a 70 wt % ethanol aqueous solution at room temperature (25° C.) for 12 hours and then filtered through 300-mesh filter cloth. The resulting extract was added to a 3 L separatory funnel and then 1 L of ethyl acetate was added. After mixing by shaking, an upper layer (ethyl acetate layer) was taken when the mixture was separated into two layers completely. The lower layer (aqueous layer) was extracted two more times using a separatory funnel. The separated upper layers were combined, concentrated under reduced pressure at 50° C. using a distillation apparatus equipped with a condenser and then dried. As a result, 10.3 g of each of soybean leaf extracts for the growth stages were obtained.

### TEST EXAMPLE 1

### Antioxidative Activity

In order to investigate the antioxidative activity of the soybean leaf extracts of stages VC, V2, V4, R2, R4, R6 and R7 prepared in Example 1, the effect of inhibiting the oxidation of the free radical 1,1-diphenyl-2-picrylhydrazyl (DPPH) was compared by measuring the change in absorbance caused by reduction of DPPH.

190 μL of a 100 μM (in ethanol) DPPH solution and each of the soybean leaf extracts of stages VC, V2, V4, R2, R4, R6 and R7 obtained in Example 1 and 10,000 ppm Trolox (positive control), a synthetic antioxidant are prepared at a concentration of 10,000 ppm, diluted to a final concentration of 500 ppm, 250 ppm, 125 ppm, 62.5 ppm, 31.25 ppm or 15.63 ppm. 10 μL of each of them were mixed to prepare a reaction solution, and absorbance was measured at 540 nm after incubation at 37° C. for 30 minutes. The analysis result is shown in Table 1. IC₅₀ means the sample concentration when the absorbance was decreased by 50% due to the added sample.

TABLE 1

| | | Ratio of concentration |
|---|---|---|
| | | required to exhibit similar |
| | | effect as stage R7 |
| | IC₅₀ | soybean leaf (autumn |
| Substances | (ppm) | soybean leaf) extract |
| Trolox (positive control) | 38 | |
| Soybean leaf extract of stage VC | No effect | X |
| Soybean leaf extract of stage V2 | No effect | X |
| Soybean leaf extract of stage V4 | 251 | 6.1 fold |
| Soybean leaf extract of stage R2 | 168 | 4.1 fold |
| Soybean leaf extract of stage R4 | 124 | 3.1 fold |
| Soybean leaf extract of stage R6 | 79 | 1.9 fold |
| Soybean leaf (autumn soybean | 41 | 1 fold |
| leaf) extract of stage R7 | | |

As can be seen from Table 1, among the soybean leaf extracts obtained in the different stages VC, V2, V4, R2, R4, R6 and R7, the extract of the soybean leaf (autumn soybean leaf) of stage R7 showed the best antioxidative activity. The extract of the soybean leaf (autumn soybean leaf) of stage R7 showed an antioxidative activity comparable to that of Trolox.

### TEST EXAMPLE 2

### Antiaging Activity

The activity of inhibiting collagenase production of the soybean leaf extracts of stages VC, V2, V4, R2, R4, R6 and R7 prepared in Example 1 was compared with those of tocopherol (Comparative Example 1) and EGCG (Comparative Example 2). Tocopherol and EGCG are known to prevent skin aging by regenerating epidermal cells.

Human fibroblasts (Cascade Biologics, Portland, Oreg., USA) were added to a 96-well microtiter plate containing Dulbecco's modified Eagle's medium (DMEM) supplemented with 2.5 wt % fetal bovine serum, at 5,000 cells/well, and cultured to about 90% confluence. Then, after culturing in serum-free DMEM for 24 hours and treating with 100 μg/mL of each of the soybean leaf extracts of stages VC, V2, V4, R2, R4, R6 and R7 prepared in Example 1 or 10⁻⁴ M tocopherol or EGCG (Sigma Aldrich) for 24 hours, the cell culture was harvested.

The production of collagenase in the cell culture harvested was measured using a collagenase measuring system (Amersham Pharmacia, USA). First, the harvested cell culture was added to a 96-well plate having primary collagenase antibodies applied uniformly thereto and an antigen-antibody reaction was carried out was incubated for 3 hours in a constant-temperature (36° C.) bath.

3 hours later, after adding chromophore-conjugated secondary collagen antibodies to the 96-well plate, incubation was performed for 15 minutes. 15 minutes later, a substance inducing color development was added and color development was induced at room temperature for 15 minutes. When 1 M sulfuric acid was added to stop the reaction (color development), the reaction solution exhibited a yellow color. The intensity of the yellow color varied depending on the degree of progress of the reaction.

The absorbance of the 96-well plate exhibiting yellow color was measured at 405 nm using a spectrophotometer and the degree of collagenase synthesis was calculated using Equation 1. A absorbance of a cell culture obtained without treating with the composition was used as a control group. That is to say, the expression level of collagenase in the group treated with the test substance was calculated relative to that of the untreated group as 100. The result is shown in Table 2.

Collagenase expression level (%)=Absorbance of group treated with test substance/Absorbance of control group×100 [Equation 1]

TABLE 2

| | Collagenase |
|---|---|
| Test substance | expression level (%) |
| Untreated group | 100 |
| Tocopherol (Comparative Example 1) | 74 |
| EGCG (Comparative Example 2) | 61 |
| Soybean leaf extract of stage VC | 98 |
| Soybean leaf extract of stage V2 | 95 |
| Soybean leaf extract of stage V4 | 92 |
| Soybean leaf extract of stage R2 | 86 |
| Soybean leaf extract of stage R4 | 81 |
| Soybean leaf extract of stage R6 | 69 |
| Soybean leaf (autumn soybean leaf) extract of | 53 |
| stage R7 | |

As can be seen from Table 2, among the soybean leaf extracts obtained in the different stages VC, V2, V4, R2, R4, R6 and R7, the extract of the soybean leaf (autumn soybean leaf) of stage R7 inhibited collagenase expression in vitro the most effectively. Its ability of inhibiting collagenase expression was superior to those of tocopherol and EGCG which are known as antiaging substances. Accordingly, it can be seen that the extract according to an aspect of the present disclosure or a composition containing the same exhibits a remarkable antiaging effect.

### TEST EXAMPLE 3

### Irritation

Retinol as a known antiaging substance and the autumn soybean leaf (stage R7) extract as an active ingredient of the present disclosure were tested comparatively for irritation by 15 panels who are sensitive to irritation such as pricking, flushing, etc.

The panels were asked to apply 0.5 mL of retinol (purchased from Sigma) or the autumn soybean leaf extract obtained in Example 1 (1 mg/mL) randomly on each face and score between 0 and 3.0 points with 0.1-point intervals. The result is shown in Table 3.

<Scoring Standard>

0-0.4: No irritation.

0.5-1.0: Slight irritation.

1.1-2.0: Moderate irritation.

2.1-3.0: Severe irritation.

TABLE 3

| | Retinol | Autumn soybean leaf extract |
|---|---|---|
| Pricking | 0.87 | 0.19 |
| Flushing | 0.42 | 0.23 |
| Mean | 0.65 | 0.21 |

As can be seen from Table 3, retinol showed slight irritation such as pricking and flushing. In contrast, the autumn soybean leaf extract according to the present disclosure showed no irritation such as pricking or flushing.

### TEST EXAMPLE 4

### Comparison of Ingredients of Autumn Soybean Leaf Extract and Soybean Extract

In order to confirm the difference in the ingredients of the autumn soybean leaf extract according to an aspect of the present disclosure and a general soybean extract, HLPC analysis was conducted on the autumn soybean leaf (stage R7) extract of Example 1 and a soybean extract obtained in Comparative Example 1.

### COMPARATIVE EXAMPLE 1

### Preparation of Soybean Extract

Soybean (Paju Jangdan soybean, purchased from Paju Nonghyup) was washed with purified water, dried and then ground into powder. 100 g of the soybean powder was extracted with 1 L of a 70 wt % ethanol aqueous solution at room temperature (25° C.) for 12 hours and then filtered through 300-mesh filter cloth. The resulting extract was added to a 3 L separatory funnel and then 1 L of ethyl acetate was added. After mixing by shaking, an upper layer (ethyl acetate layer) was taken when the mixture was separated into two layers completely. The lower layer (aqueous layer) was extracted two more times using a separatory funnel. The separated upper layers were combined, concentrated under reduced pressure at 50° C. using a distillation apparatus equipped with a condenser and then dried. As a result, 13.4 g of a soybean extract was obtained.

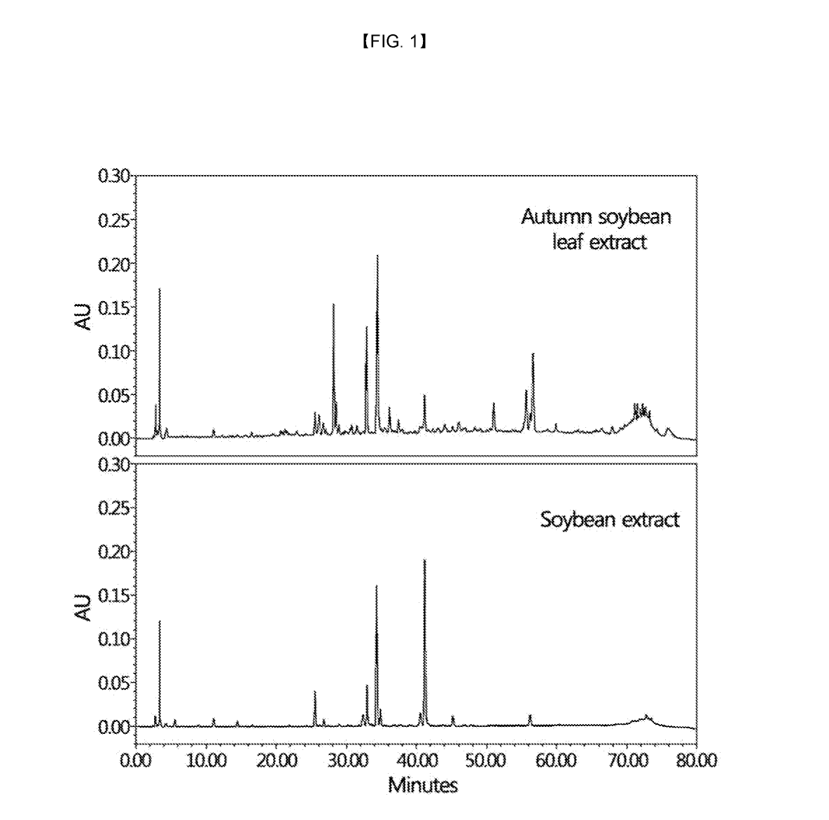

The analysis of ingredients (Waters, 2996 PDA detector) was conducted by using HPLC (Waters, Model 2695) after dissolving the soybean extract and the autumn soybean leaf extract in 70 vol % ethanol to make 10,000 ppm solutions. Kanto Chemical's Mightysil RP-18 GP 250-4.6 (5 μm) column was used as a stationary phase and the compositions described in Table 4 were used as a mobile phase. The result is shown in FIG. 1.

TABLE 4

| Time (min) | Water (0.1% acetic acid) | Acetonitrile (0.1% acetic acid) |
|---|---|---|
| 0 | 95 | 5 |
| 65 | 62 | 38 |
| 70 | 38 | 62 |
| 75 | 95 | 5 |
| 80 | 95 | 5 |

From FIG. 1, the autumn soybean leaf extract according to an aspect of the present disclosure and the soybean extract of Comparative Example 1 showed entirely different HPLC results. They showed difference in the number and intensity of HPLC peaks. Accordingly, it can be seen that the autumn soybean leaf extract and the soybean extract having different properties contain different ingredients and it is expected that they will exhibit different antioxidative and antiaging effects.

Hereinafter, formulation examples of the composition according to an aspect of the present disclosure are described. They are intended to illustrate the present disclosure and are not intended to limit the present disclosure.

### FORMULATION EXAMPLE 1

### Milk Lotion (Nourishing Lotion)

A nourishing lotion was prepared according to a common method with the composition described in Table 5.

TABLE 5

| Ingredients | wt % |
|---|---|
| Autumn soybean leaf extract of Example 1 | 3.00 |
| L-Ascorbic acid 2-phosphate magnesium salt | 1.00 |
| Water-soluble collagen (1% aqueous solution) | 1.00 |
| Sodium citrate | 0.10 |
| Citric acid | 0.05 |
| Celandine extract | 0.20 |
| 1,3-Butylene glycol | 3.00 |
| Purified water | to 100 |

### FORMULATION EXAMPLE 2

### Nourishing Cream

A nourishing cream was prepared according to a common method with the composition described in Table 6.

TABLE 6

| Ingredients | wt % |
|---|---|
| Autumn soybean leaf extract of Example 1 | 1.00 |
| Polyethylene glycol monostearate | 2.00 |
| Self-emulsifying glyceryl monostearate | 5.00 |
| Cetyl alcohol | 4.00 |
| Squalane | 6.00 |
| Tri-2-ethylhexanoate glyceryl | 6.00 |
| Sphingoglycolipid | 1.00 |
| 1,3-Butylene glycol | 7.00 |
| Purified water | to 100 |

### FORMULATION EXAMPLE 3

### Pack

A pack was prepared according to a common method with the composition described in Table 7.

TABLE 7

| Ingredients | wt % |
|---|---|
| Autumn soybean leaf extract of Example 1 | 5.00 |
| Polyvinyl alcohol | 13.00 |
| L-Ascorbic acid 2-phosphate magnesium salt | 1.00 |
| Lauroyl hydroxyproline | 1.00 |
| Water-soluble collagen (1% aqueous solution) | 2.00 |
| 1,3-Butylene glycol | 3.00 |
| Ethanol | 5.00 |
| Purified water | to 100 |

### FORMULATION EXAMPLE 4

### Beauty Solution

A beauty solution was prepared according to a common method with the composition described in Table 8.

TABLE 8

| Ingredients | wt % |
|---|---|
| Autumn soybean leaf extract of Example 1 | 2.00 |
| Hydroxyethyl cellulose (2% aqueous solution) | 12.00 |
| Xanthan gum (2% aqueous solution) | 2.00 |
| 1,3-Butylene glycol | 6.00 |
| Thick glycerin | 4.00 |
| Sodium hyaluronate (1% aqueous solution) | 5.00 |
| Purified water | to 100 |

### FORMULATION EXAMPLE 5

### Ointment

An ointment was prepared according to a common method with the composition described in Table 9.

TABLE 9

| Ingredients | wt % |
|---|---|
| Autumn soybean leaf extract of Example 1 | 0.1 |
| Glycerin | 8.0 |
| Butylene glycol | 4.0 |
| Liquid paraffin | 15.0 |
| β-Glucan | 7.0 |
| Carbomer | 0.1 |
| Caprylic/capric triglyceride | 3.0 |
| Squalane | 1.0 |
| Cetearyl glucoside | 1.5 |
| Sorbitan stearate | 0.4 |
| Cetearyl alcohol | 1.0 |
| Beeswax | 4.0 |
| Antiseptic, pigment and flavor | Adequate |
| Purified water | Balance |

### FORMULATION EXAMPLE 6

### Soft Capsule

A soft capsule filling solution was prepared by mixing 8 mg of the autumn soybean leaf extract of Example 1, 9 mg of vitamin E, 9 mg of vitamin C, 2 mg of palm oil, 8 mg of hydrogenated vegetable oil, 4 mg of yellow beeswax and 9 mg of lecithin according to a common method. A soft capsule was prepared by filling 400 mg of the solution per capsule. Separately from this, a soft capsule sheet was prepared from 66 parts by weight of gelatin, 24 parts by weight of glycerin and 10 parts by weight sorbitol solution and the filling solution was filled therein to prepare a soft capsule containing 400 mg of the composition according to an aspect of the present disclosure.

### FORMULATION EXAMPLE 7

### Tablet

8 mg of the autumn soybean leaf extract of Example 1, 9 mg of vitamin E, 9 mg of vitamin C, 200 mg of galactooligosaccharide, 60 mg of lactose and 140 mg of maltose were mixed and granulated using a fluidized-bed dryer. After adding 6 mg of sugar ester, 500 mg of the resulting composition was prepared into a tablet according to a common method.

### FORMULATION EXAMPLE 8

### Drink

8 mg of the autumn soybean leaf extract of Example 1, 9 mg of vitamin E, 9 mg of vitamin C, 10 g of glucose, 0.6 g of citric acid and 25 g of oligosaccharide syrup were mixed and 300 mL of purified water was added. 200 mL of the mixture was filled per bottle and sterilized at 130° C. for 4-5 seconds to prepare a drink.

### FORMULATION EXAMPLE 9

### Granule

8 mg of the autumn soybean leaf extract of Example 1, 9 mg of vitamin E, 9 mg of vitamin C, 250 mg of anhydrous crystalline glucose and 550 mg of starch were mixed and granulated using a fluidized-bed granulator. The resulting granule was filled in a pouch to prepare a granule.

### FORMULATION EXAMPLE 10

### Injection

An injection was prepared according to a common method with the composition described in Table 10.

TABLE 10

| Ingredients | Contents |
|---|---|
| Autumn soybean leaf extract of Example 1 | 10-50 mg |
| Sterilized distilled water for injection | Adequate |
| pH control agent | Adequate |

### FORMULATION EXAMPLE 11

### Health Functional Food

A health functional food was prepared according to a common method with the composition described in Table 11.

TABLE 11

| Ingredients | Contents | |
|---|---|---|
| Autumn soybean leaf extract of Example 1 | 20 | mg |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The compositional ratios of the vitamin and mineral mixtures described above, which are given as a specific example relatively suitable for a health functional food, may be varied as desired.

### FORMULATION EXAMPLE 12

### Health Drink

A health drink was prepared according to a common method with the composition described in Table 12.

TABLE 12

| Ingredients | Contents | |
|---|---|---|
| Autumn soybean leaf extract of Example 1 | 1000 | mg |
| Citric acid | 1000 | mg |
| Oligosaccharide | 100 | g |
| Taurine | 1 | g |

| | |
|---|---|
| Purified water | Balance |

According to a common health drink preparation method, the above ingredients were mixed and heated at 85° C. for about 1 hour under stirring. The resulting solution was filtered and sterilized.

While the exemplary embodiments have been shown and described, it will be understood by those skilled in the art that various changes in form and details may be made thereto without departing from the spirit and scope of this disclosure as defined by the appended claims.

1-13. (canceled)

14. A composition for external application to skin, which comprises an autumn soybean leaf extract as an active ingredient.

15. The composition according to claim 14, wherein the autumn soybean leaf is the leaf of soybean in growth stages R7 to R8.

16. The composition according to claim 14, wherein the composition comprises 0.001-20 wt % of the autumn soybean leaf extract based on the total weight of the composition.

17. The composition according to claim 14, wherein the autumn soybean leaf extract is an extract of one or more selected from a group consisting of water, an organic solvent and a mixture thereof.

18. The composition according to claim 17, wherein the organic solvent is one or more selected from a group consisting of a C₁-C₆ lower alcohol, acetone, ether, ethyl acetate, diethyl ether, ethyl methyl ketone and chloroform.

19. The composition according to claim 18, wherein the autumn soybean leaf extract is an extract extracted sequentially with ethanol and ethyl acetate.

20. The composition according to claim 14, wherein the composition inhibits the oxidation of 1,1-diphenyl-2-picrylhydrazyl (DPPH).

21. The composition according to claim 14, wherein the composition inhibits the expression of collagenase.

22. The composition according to claim 14, wherein the autumn soybean leaf extract is obtained by a method comprising: extracting autumn soybean leaf first with water, an organic solvent or a mixture thereof; extracting second by adding an organic solvent to the resulting extract; and concentrating the resulting extract under reduced pressure and then drying the same.

23. The composition according to claim 14, wherein the autumn soybean leaf extract has one or more of the following characteristics:
(1) IC₅₀≦70 ppm in antioxidative activity test based on DPPH reduction; and
(2) collagenase expression level≦55% in collagenase production inhibition test.

24. A method for antioxidating or antiaging, which comprises administering an effective amount of an autumn soybean leaf extract to a subject in need thereof.

25. The method according to claim 24, wherein the autumn soybean leaf is the leaf of soybean in growth stages R7 to R8.

26. The method according to claim 24, wherein the autumn soybean leaf extract is administered in a form of a composition, and the composition comprises 0.001-20 wt % of the autumn soybean leaf extract based on the total weight of the composition.

27. The method according to claim 24, wherein the autumn soybean leaf extract is an extract of one or more selected from a group consisting of water, an organic solvent and a mixture thereof.

28. The method according to claim 27, wherein the organic solvent is one or more selected from a group consisting of a C₁-C₆ lower alcohol, acetone, ether, ethyl acetate, diethyl ether, ethyl methyl ketone and chloroform.

29. The method according to claim 28, wherein the autumn soybean leaf extract is an extract extracted sequentially with ethanol and ethyl acetate.

30. The method according to claim 24, wherein the autumn soybean leaf extract is administered in a form of a composition, and the composition inhibits the oxidation of 1,1-diphenyl-2-picrylhydrazyl (DPPH).

31. The method according to claim 24, wherein the autumn soybean leaf extract is administered in a form of a composition, and the composition inhibits the expression of collagenase.

32. The method according to claim 24, wherein the autumn soybean leaf extract is obtained by a method comprising: extracting autumn soybean leaf first with water, an organic solvent or a mixture thereof; extracting second by adding an organic solvent to the resulting extract; and concentrating the resulting extract under reduced pressure and then drying the same.

33. The method according to claim 24, wherein the autumn soybean leaf extract has one or more of the following characteristics:
(1) IC₅₀≦70 ppm in antioxidative activity test based on DPPH reduction; and
(2) collagenase expression level≦55% in collagenase production inhibition test.
